(19) United States
(12) Patent Application Publication
(10) Pub. No.: US 2017/0027515 A1
(43) Pub. Date: Feb. 2, 2017

(54) Non-Invasive Continuous Monitoring of Physiological Parameters

(71) Applicant: Verily Life Sciences LLC, Mountain View, CA (US)

(72) Inventor: Robert Francis Wiser, San Francisco, CA (US)

(21) Appl. No.: 14/813,713

(22) Filed: Jul. 30, 2015

(51) Int. Cl. A61B 5/00; A61B 5/145; A61B 5/0205

(52) U.S. Cl. CPC A61B 5/6833; A61B 5/0059; A61B 5/02055; A61B 5/14542; A61B 2562/0219

(57) ABSTRACT

Systems and methods are described that relate to a body-mountable device. The body-mountable device includes a first light source and a second light source, which may emit light having at least a first and second wavelength, respectively. The body-mountable device includes at least one sensor configured to detect light having the first wavelength and the second wavelength. The body-mountable device includes a battery, a communication interface, and a controller. The controller includes a memory, a processor, and a sigma-delta analog-to-digital converter (ADC) having one or more channels. The controller performs various operations, such as causing the first light source and the second light sources to emit light and receiving information indicative of at least one physiological parameter via the at least one sensor. The controller may also perform processing, via the sigma delta ADC, the information indicative of the at least one physiological parameter so as to provide physiological data.

### BACKGROUND

Unless otherwise indicated herein, the materials described in this section are not prior art to the claims in this application and are not admitted to be prior art by inclusion in this section.

Certain medical conditions or states can be characterized by a physiological property over long periods of time and/or by infrequent, short-timescale events. Such physiological properties can be measured periodically. An implanted or wearable device could be employed to provide continuous or near-continuous measurement of such physiological properties. Such implantable or wearable devices can be battery-powered and/or powered by radio frequency energy or other wireless energy sources. Further, such devices can be configured to indicate measured physiological properties wirelessly.

### SUMMARY

In a first aspect, a system is provided. The system includes a body-mountable device and a mobile computing device. The body-mountable device is configured as an adhesive patch. The body-mountable device includes a first light source and a second light source. The first light source is configured to emit light having at least a first wavelength and the second light source is configured to emit light having at least a second wavelength. The body-mountable device further includes at least one sensor. The at least one sensor includes one or more photodetectors configured to detect light having the first wavelength and to detect light having the second wavelength. The body-mountable device also includes a battery, a communication interface, and a controller. The controller includes a memory and a processor. The memory stores instructions that are executable by the processor to cause the controller to perform operations. The operations include causing the first light source and the second light source to emit light and receiving information from the at least one sensor. The information is indicative of at least one physiological parameter. The operations also include transmitting, via the communication interface, the information indicative of at least one physiological parameter. The mobile computing device is configured to receive the transmitted information indicative of at least one physiological parameter.

In a second aspect, a body-mountable device is provided. The body-mountable device configured as an adhesive patch. The body-mountable device comprises a first light source and a second light source. The first light source is configured to emit light having at least a first wavelength and the second light source is configured to emit light having at least a second wavelength. The body-mountable device also includes at least one sensor. The at least one sensor includes one or more photodetectors configured to detect light having the first wavelength and to detect light having the second wavelength. The body-mountable device also includes a battery and a communication interface. The communication interface includes at least one of: a BLUETOOTH Low Energy (BLE) interface, a radio frequency identification (RFID) interface, or a near-field communication (NFC) interface. The body-mountable device additionally includes a controller including a memory, a processor, and a sigma-delta analog-to-digital converter (ADC) having one or more data channels. The memory stores instructions that are executable by the processor to cause the controller to perform operations. The operations include causing the first light source and the second light source to emit light. The operations further include receiving information from the at least one sensor. The information is indicative of at least one physiological parameter. The operations additionally include processing, via the one or more data channels of the sigma-delta analog-to-delta converter (ADC), the information indicative of the at least one physiological parameter so as to provide physiological data. The operations yet further include storing the physiological data in the memory and wirelessly transmitting the physiological data via the communication interface.

In a third aspect, a method is provided. The method includes causing a first light source to emit light having a first wavelength and a second light source to emit light having a second wavelength. The first and second light sources are in a body-mountable device. The body-mountable device is configured as an adhesive patch. The method includes detecting, by at least one sensor in the body-mountable device, information indicative of at least one physiological parameter. The at least one sensor includes one or more photodetectors configured to detect light having the first wavelength and to detect light having the second wavelength. The method includes processing, via one or more data channels of a sigma-delta analog-to-digital converter (ADC), the information indicative of the at least one physiological parameter so as to provide physiological data. The method includes storing the physiological data in memory and wirelessly transmitting the physiological data via a communication interface. The communication interface includes at least one of: a BLUETOOTH Low Energy (BLE) interface, a radio frequency identification (RFID) interface, or a near-field communication (NFC) interface.

Other aspects, embodiments, and implementations will become apparent to those of ordinary skill in the art by reading the following detailed description, with reference where appropriate to the accompanying drawings.

### BRIEF DESCRIPTION OF THE FIGURES

FIG. 1 is a block diagram of a system according to an example embodiment.

FIG. 2 illustrates a body-mountable device according to an example embodiment.

FIG. 3A illustrates a body-mountable device according to an example embodiment.

FIG. 3B illustrates a body-mountable device according to an example embodiment.

FIG. 4A illustrates a mobile device according to an example embodiment.

FIG. 4B illustrates a mobile device according to an example embodiment.

FIG. 5 is a flowchart illustrating a method according to an example embodiment.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are contemplated herein.

Further, while embodiments disclosed herein make reference to use on or in conjunction with a living human body, it is contemplated that the disclosed methods, systems and devices may be used in any environment where measuring and/or estimating a flow rate of a liquid, such as blood, is desirable. The environment may be any living or non-living body or a portion thereof, a fluid conduit, a fluid reservoir, etc. For example, one of skill in the art will recognize that the embodiments disclosed herein may be used to sense the flow rate of water or another liquid. Moreover, while the present disclosure describes embodiments for use in vivo, one of skill in the art will also recognize that in vitro applications are possible as well. Accordingly, the environment may also include a test tube or other vessel for holding a fluid.

Additionally, the term “medical condition” as used herein should be understood broadly to include any disease, illness, disorder, injury, condition or impairment—e.g., physiologic, psychological, cardiac, vascular, orthopedic, visual, speech, or hearing—or any situation requiring medical attention.

### Overview

Systems, devices, and methods described herein may relate to continuous monitoring of physiological parameters of a living body. Specifically, the continuous monitoring may be carried out via a body-mountable device, which may be in the form of a small adhesive patch.

The body-mountable device may include a plurality of light sources and at least one detector operable to detect a physiological parameter via a photoplethysmography (PPG) technique. For example, the body-mountable device may include a first light source and a second light source, which may be operable to emit light at a first wavelength and a second wavelength, respectively.

The body-mountable device may also include a battery, a communication interface, and a controller. The battery may be a thin film battery, which may be operable to power the body-mountable device for one week, or longer. The communication interface may include a BLUETOOTH Low Energy (BLE) interface or a near-field communication (NFC) interface. The controller may include a memory and a processor. The controller may store instructions in the memory and the processor may be operable to execute the instructions. As such, the controller may be operable to perform various operations, which may relate to obtaining, processing, and transmitting information indicative of a physiological parameter of a wearer of the body-mountable device.

The operations may include causing the first light source and the second light source to emit light and detecting light via the at least one detector. The light sources and detector may function together as a PPG sensor. The detected light may be used to obtain information about the wearer's pulse rate, blood pressure, etc.

The operations may further include processing signals from the PPG sensor(s) so as to form physiological data. For example, in an example embodiment, the body-mountable device may include a sigma-delta analog-to-digital converter (ADC) with one or more data channels. In such a scenario, the ADC may process the signals from the PPG sensor(s) via the one or more data channels so as to form physiological data.

Optionally, the body-mountable device may be configured to obtain physiological data via additional sensors. For example, a respiratory rate can be determined by including information from an accelerometer, and skin temperature (or core body temperature) may be determined via an external thermistor or an implanted temperature sensor.

The body-mountable device may include a small form factor (e.g. a circular patch with 1.5″ diameter and less than 2 mm thick), which may allow the device to be worn at the same level of the heart, reducing effects of posture and movement on the PPG signal and simplifying the estimation of blood pressure. The body-mountable device may include a bio-compatible package. For instance, the body-mountable device may be packaged and/or encapsulated in a flexible polyimide and/or parylene material. Furthermore, while some embodiments herein describe an adhesive patch, other embodiments may include the body-mountable device configured to be mounted via a strap, an implant, etc. Additionally or alternatively, the body-mountable device may be mounted anywhere on or within the living body, such as on the wrist, torso, ankle, neck, etc.

The physiological parameters contemplated herein may include at least one of: a heart rate, a blood pressure, or a blood oxygenation level. Other physiological parameters that may be detected via a body-mountable device, particularly an adhesive patch, are considered within the scope of this disclosure as well.

The system may also include a mobile computing device. The mobile computing device may be a handheld reader, a smartphone, a tablet, a smartwatch, a laptop, or another type of mobile computing device. The mobile computing device may be configured to be communicatively coupled to the body-mountable device. For example, the mobile computing device may receive the information indicative of the at least one physiological parameter via the communication interface.

The mobile computing device may activate the body-mountable device to start data collection, to download the last time period of data, and disable the body-mountable device. The time period of data may range from seconds to hours, depending on sample rate and memory size, with new data overwriting the oldest values. The physiological data may be formatted such that the data has a relative or absolute time stamp. For example, the mobile computing device may be configured to determine a time stamp based on an integrated circuit clock frequency and sample rate.

Processing of the raw data may be performed at the body-mountable device or at the mobile computing device. The processed raw data may be termed physiological data and may include a ratiometric determination of blood oxygenation, pulse rate from based on data periodicity, blood pressure from PPG data slopes, and respiratory rate from a combination of accelerometer data and pulse rate.

The systems, devices, and methods described herein may be useful to provide long-term monitoring of physiological parameters. Such monitoring may provide more accurate and/or timelier health information for wearers of the body-mountable device. Furthermore, if elected by the wearer of the body-mountable device, health care professionals may be kept continuously or periodically apprised of the health information so as to provide better health care for patients. Additionally, if the body-mountable device or a combination of the body-mountable device and the mobile computing device detects an emergency situation, emergency personnel may be alerted of the health condition and location of the wearer of the sensor. System Examples

Some examples of the present disclosure relate to systems and devices that are configured to be worn on a living body and that may be operable to obtain measurements related to physiological conditions of the body via sensors on the devices. Such devices, described herein as body-mountable devices, can be configured to be mounted to an external body surface of a wearer and to enable a variety of applications and functions. The terms “body-mountable device” or “wearable device,” as used in this disclosure, refers to any device that is capable of being worn at, on or in proximity to a body surface, such as a shoulder, forearm, wrist, ankle, waist, chest, back, buttocks, or another body part. In an example embodiment, the body-mountable device is configured as an adhesive patch.

One or more sensors disposed on a portion of the contact surface can detect one or more properties of the body of the wearer when the contact surface is mounted to the external body surface. The body-mountable device may also include a controller configured to receive, store, and transmit information indicative of a physiological parameter of the wearer.

Such body-mountable devices could enable a variety of applications related to the physiological information about a wearer. For example, a mobile device associated with the body-mountable device may be configured to indicate such measured physiological information or other information to the wearer (e.g., using a haptic feedback system, a display system, an audio feedback system, a communication to a server that is configured to generate an indication to the wearer, or other user interface systems).

An example body-mountable device can automatically measure multiple physiological parameters of a person wearing the device via sensors included in the device. In order to take measurements in a non-invasive manner from outside of the body, the body-mountable device may be positioned on a portion of the body where subsurface vasculature or other elements of the body of the wearer may be detected. Thus, the body-mountable device may be placed in close proximity to the skin or tissue. For example, the body-mountable device may be configured to adhere to a skin layer of a living body. Depending on the mounting location of the device, the device may accordingly be implemented in a variety of different form factors that are configured to be mounted to a variety of different body surfaces.

The devices and systems described herein may provide continuous monitoring of vital signs with a single noninvasive body-mountable device. In an example embodiment, the body-mountable device may remain mounted to a skin surface for about one week, which may provide wearers and physicians with a rich data set of physiological parameters. Long-term monitoring of such physiological parameters may provide an indication as to a person's present overall health, and may provide information useful to help improve or maintain a person's health in the future.

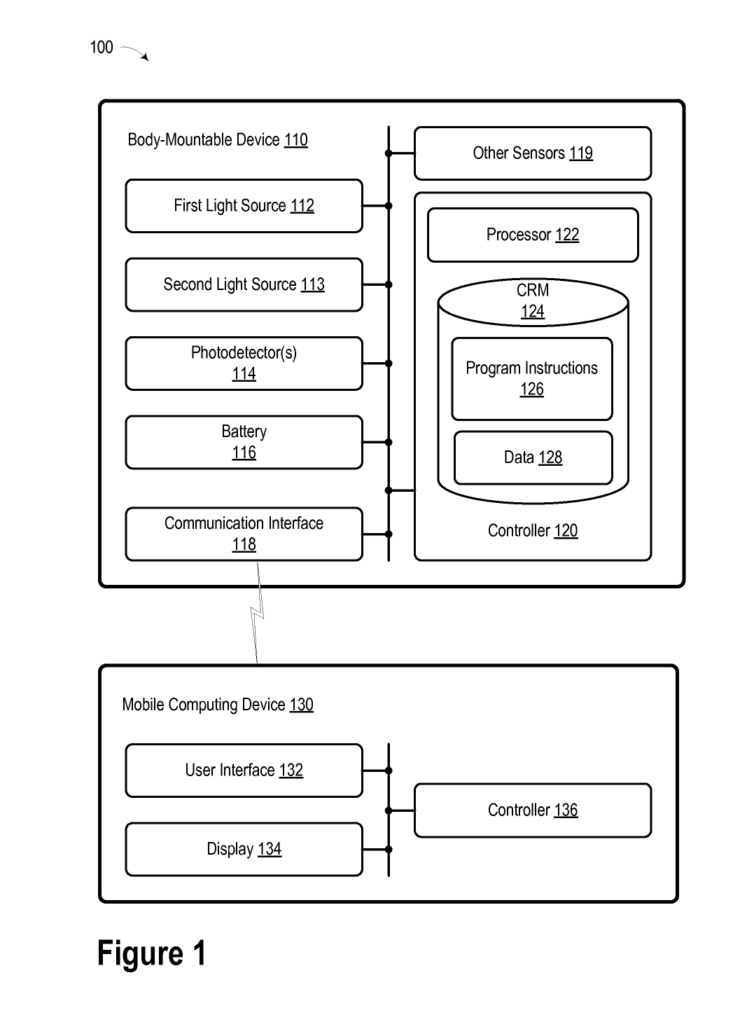

FIG. 1 is a block diagram of a system 100 according to an example embodiment. System 100 includes a body-mountable device 110 and a mobile computing device 130. The body-mountable device 110 includes a first light source 112 and a second light source 113. The first light source 112 and the second light source 113 may include a light-emitting diode, a vertical-cavity emitting laser (VCSEL), a ridge waveguide laser, or another type of light source. The first light source 112 and the second light sources 113 may be configured to emit over respective emission wavelengths that are at least partially non-overlapping. That is, the first light source 112 and the second light source 113 may be configured to emit light at different wavelengths. In an example embodiment, the first light source 112 may be configured to emit light at a first wavelength. The first wavelength may include a wavelength in the infrared waveband (e.g. between 750 nanometers and 1 millimeter). The second light source 113 may be configured to emit light at a second wavelength. The second wavelength may include a wavelength in the red portion of the visible light spectrum (e.g. between 620 nanometers and 750 nanometers). The first and the second light sources may be configured to emit within the above-described wavelength ranges to provide pulse oximetry information, however other wavelengths and wavelength ranges are possible.

In a further example embodiment, at least one of the light sources may be configured to emit in the green portion of the visible light spectrum (e.g. between 495 nanometers and 570 nanomters). In such a scenario, the system 100 may be configured to provide photoplethysmogram information. It is understood that these emission wavelengths are provided as examples and that many other wavelengths and/or wavebands are possible within the scope of the present disclosure. Furthermore, one or more of the light sources may be configured to be tunable over a range of emission wavelengths and/or emission wavebands.

The body-mountable device 110 additionally includes one or more photodetectors 114 that are configured to detect light from the first light source 112 and/or the second light source 113. In some embodiments, a single photodetector may be configured to detect light from both the first light source 112 and the second light source 113. Thus, the photodetector 114 may be configured to detect light having the first wavelength and the second wavelength. In an example embodiment, the one or more photodetectors 114 are mounted proximate to an exterior surface of the living body so as to receive light transmitted through and/or reflected by the living body from the first light source 112 and the second light source 113. The one or more photodetectors 114 may include photodiodes, photoconductors, cameras, or another type of light-sensitive device. In an example embodiment, the one or more photodetectors 114 are configured to sample light at the first wavelength and/or the second wavelength at a rate of at least 120 samples per second. Higher or lower sampling rates are possible.

In an example embodiment, the one or more photodetectors 114 may be configured to provide data about light transmission through or reflection from a lumen of a living body. For example, as described above, the light sources and one or more photodetectors 114 may be operable to provide physiological information via a photoplethysmography (PPG) technique. In such a scenario, the intensity, polarization, or another characteristic of transmitted or reflected light may vary depending on a volume of fluid, e.g. blood, in the lumen. Over time, the volume of fluid in the lumen may change, which may indicate a pulse rate. Furthermore, a blood pressure, pulse oximetry, and/or a flow rate of the fluid through the lumen may be estimated and/or inferred.

The body-mountable device 110 also includes a battery 116, a communication interface 118, an antenna (not illustrated) and other sensors 119. The battery 116 may include a primary (non-rechargeable) or a secondary (rechargeable) battery. In an example embodiment, the battery 116 may include a thin film cell battery. Furthermore, in an example embodiment, battery 116 may include an alkaline battery, a nickel-metal-hydride battery or a lithium-ion battery. Other types of batteries are possible.

The communication interface 118 may be operable to establish a communication link with the mobile computing device 130 via one or more communication protocols. For example, the communication interface 118 may be configured to communicate with the mobile computing device 130 via a BLUETOOTH LOW-ENERGY (BLE), ZIGBEE, and/or Wi-Fi communication protocol. Additionally or alternatively, the communication interface 118 may be configured to establish the communication link with the mobile computing device 130 via near-field communication (NFC) and/or radio-frequency identification (RFID). Other types of communication protocols are possible and contemplated herein.

The communication interface 118 may include hardware and/or software configured to enable wireless communication between the body-mountable device 110 and other devices. For example, the communication interface 118 may enable messaging and/or data transfers between the body-mountable device 100, the mobile computing device 130, and/or a cloud-computing server.

While FIG. 1 illustrates one body-mountable device 110 as being communicatively-coupled to one mobile computing device 130, it is understood that any number of body-mountable devices 110 may be communicatively-coupled to any number of mobile computing devices 130. For example, a plurality of body-mountable devices 110 may be mounted via a skin surface of a living body. Each of the plurality of body-mountable devices 110 may be communicatively-coupled to one or more mobile computing devices 130 that may interrogate and/or monitor information obtained by the body-mountable devices 110. As such, an individual may be able to obtain more reliable and/or accurate information relating to one or more physiological parameters.

The body-mountable device 110 may optionally include other sensors 119. The other sensors 119 may include an accelerometer, a temperature sensor, or a piezoresistor. Other sensors configured to obtain information about a physiological parameter of a living body are contemplated herein. It is understood that the other sensors 119 may include a variety of different biometric sensors that may be operable to provide electrical, chemical, and/or mechanical information about a physiological parameter of a living body. The physiological parameter may include, but is not limited to, a heart rate, a blood pressure, a blood flow rate, a body temperature, a galvanic skin response measurement, or a blood oxygenation level. Other types of physiological parameters are contemplated.

The body-mountable device 110 includes a controller 120, which may include a processor 122 and a computer-readable medium (CRM) 124. The CRM 124 may include program instructions 126 and/or data storage 128. Controller 120 may include one or more computers, a computing network, and/or a cloud computing server.

In an example embodiment, the body-mountable device 110 and/or the controller 120 may include a sigma-delta analog to digital converter (ADC). The sigma-delta ADC may encode analog signals, such as those from the one or more photodetectors 114 and/or other sensors 119, into digital signals. The analog signals may be encoded as a stream of current and/or voltage pulses. Specifically, the analog signals may be encoded as a pulse frequency proportional to the analog input voltage. When decoded accurately, the analog signal may be recovered almost noiselessly from the digital signal using the sigma-delta modulation technique. The sigma-delta ADC may be implemented via hardware or software or a combination of hardware and software. In an example embodiment, the sigma-delta ADC may include one or more channels, e.g. output channels. Each channel of the sigma-delta ADC may be assigned and/or associated with one or more sensors of the body-mountable device 110. In the scenario where the sigma-delta ADC includes a plurality of channels, information from multiple sensors may be simultaneously digitized and/or transmitted from the body-mountable device 110. In a further example embodiment, multiple sigma-delta ADCs may be included in the body-mountable device 110 and/or the controller 120.

The mobile computing device 130 may include a user interface 132, a display 134, and a controller 136. The user interface 132 may include a touch screen, touch pad, and/or one or more buttons. In an example embodiment, the user interface 132 may provide a way to interact with the mobile computing device 130 and/or the body-mountable device 110. The display 134 may be operable to provide notifications, information, and/or options to a user of the mobile computing device 130.

The controller 136 of the mobile computing device 130 may include a processor and a memory. The controller 136 may be operable to execute operations on the mobile computing device 130. For example, the controller 136 of the mobile computing device 130 may be operable to receive data from the body-mountable device 110, store the data, and further transmit the data to correspondent computing devices, such as a cloud network server. The controller 136 may be configured to process some or all of the data received from the body-mountable device 110. In an example embodiment, the controller 136 may include a sigma delta analog-to-digital converter (ADC). Furthermore, various signal processing functions (e.g. summing, denoising, amplification, etc.) may be provided by the controller 136. Furthermore, the controller 136 may be configured to infer, predict, measure, and/or calculate various physiological parameters as described herein.

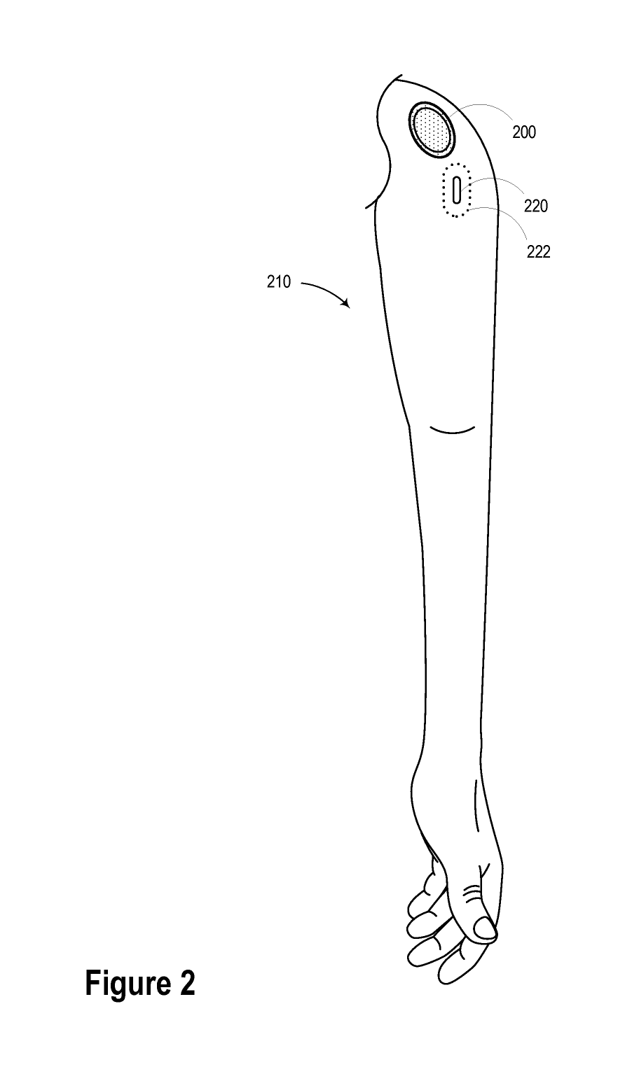

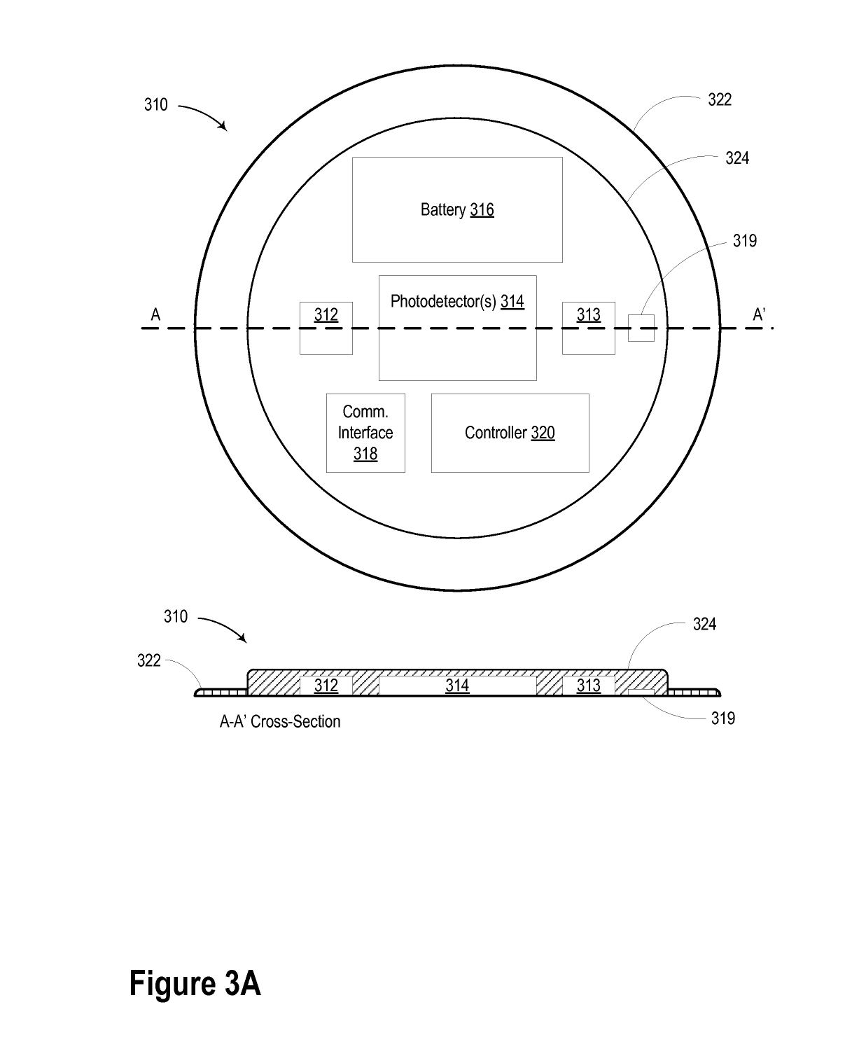

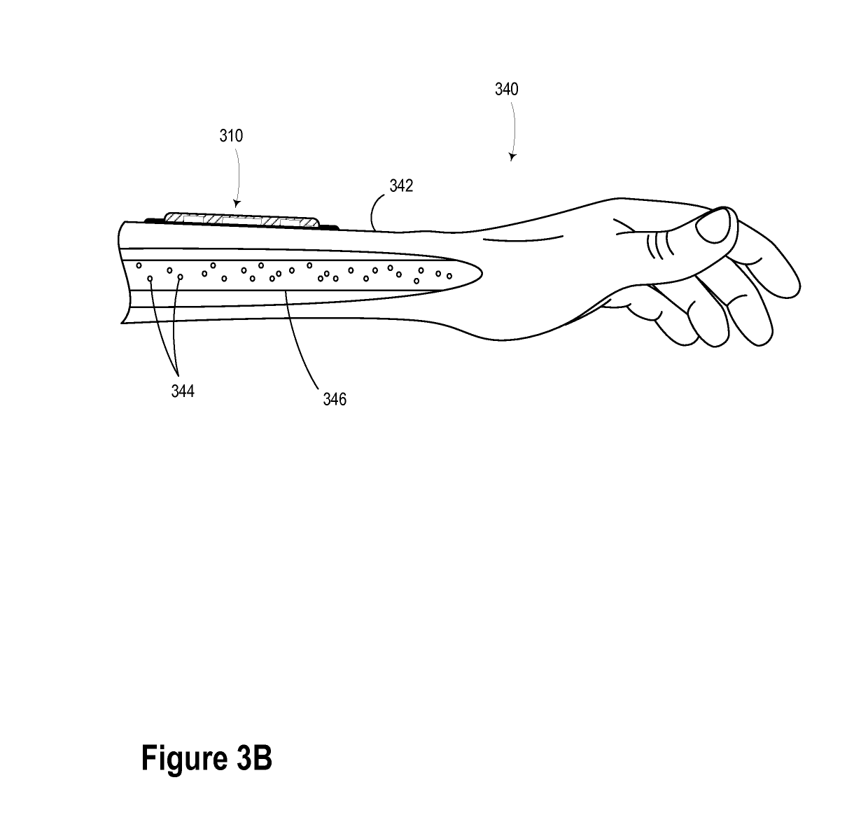

Examples of body-mountable devices, which may include the body-mountable device 100, are now described in reference to FIGS. 2, 3A, and 3B. FIG. 2 illustrates a body-mountable device 200 according to an example embodiment. In an example embodiment, the body-mountable device 200 may be configured and/or implemented as an adhesive patch. The adhesive patch may be mounted via adhesive to a skin surface of a living body 210. Namely, the body-mountable device 200 may be mounted on a shoulder or another location on the living body 210. In an example embodiment, the body-mountable device 200 may be mounted at or near the level of the heart, which may reduce the effects of body motion on measured physiological parameters (e.g. blood pressure). The body-mountable device 200 may include a housing that is biocompatible and/or hypoallergenic. For example, the housing of the body-mountable device 200 may include one or more of parylene, polyimide, polyethylene, polycarbonate, or polymethylmethacrylate (PMMA). Other materials are possible.

The body-mountable device 200 may be associated and/or communicatively coupled with other sensors. For example, body-mountable device 200 may be communicatively coupled to thermal sensor 220. Thermal sensor 220 may be a thermistor implanted in the skin at an implant location 222, which may be at a different location than the body-mountable device 200. Additionally or alternatively, the other sensors may be collocated with and/or integrated into the body-mountable device 200. The body-mountable device 200 and the other sensors may be communicatively coupled via BLUETOOTH or another communication protocol.

FIG. 3A illustrates a body-mountable device 310 according to an example embodiment. The body-mountable device 310 may be similar or identical to body-mountable devices 110 and 200 illustrated and described in reference to FIGS. 1 and 2. For example, the body-mountable device 310 may be configured as an adhesive patch and may be mountable on a skin surface of a living body. Mounting the body-mountable device 310 proximate to the heart, for example on a skin surface of the chest, shoulder, or back, may reduce motion artifacts compared to other PPG measurements obtained from extremities, such as from a finger.

The body-mountable device 310 includes a first light source 312 and a second light source 313. The body-mountable device 310 includes one or more photodetectors 314 and a battery 316. The body-mountable device 310 also includes a communication interface 318 and a controller 320.

As described elsewhere herein, the first light source 312, the second light source 313, and the one or more photodetectors 314 may be operable as elements of a multi-color photoplethysmography (PPG) system. Namely, the first light source 312 may be configured to emit light having a first wavelength and the second light source 313 may be configured to emit light having a second wavelength. The combination of multiple wavelength light sources and the one or more photodetectors 314 may provide optical information indicative of a volume of a subcutaneous region of the living body, e.g. subsurface vasculature. For example, the optical information may relate to a volume of blood within the subcutaneous region. The optical information may be processed in various ways to measure, calculate, and/or infer a variety of physiological parameters.

In an example embodiment, a blood oxygenation level may be obtained via a ratiometric comparison between the multiple wavelengths received via the one or more photodetectors. In another embodiment, a pulse rate may be obtained via a periodicity of the optical information. In yet another embodiment, blood pressure may be calculated and/or inferred via a time-dependent slope (e.g. derivative) of the periodic optical information. Additionally or alternatively, a respiratory rate may be obtained via a combination of accelerometer data and pulse rate. Other types of physiological parameters are contemplated herein.

While FIG. 3A illustrates the first light source 312 and the second light source 313 as being spatially separated, some embodiments may include multiple light sources that are configured to emit light along the same optical path. For example, light emitted from the first and second light sources 312 and 313 may be coupled into an optical fiber, which may provide a collinear optical path for the light to enter the skin surface. It is understood that various other optical elements may be incorporated into the body-mountable device 310 so as to form a multi-color PPG system. For example, the other optical elements may include, but are not limited to, spectral filters, lenses, collimators, diffusers, mirrors, gratings, splitters, and optical fibers.

The body-mountable device 310 may include other sensors 319. For example, the other sensor 319 may include an accelerometer configured to provide information about a movement of the living body.

In some embodiments, the body-mountable device 310 may include a main body portion 324 and an adhesive portion 322. For example, the main body portion 324 may house and/or encapsulate the electronic and/or mechanical elements of the body-mountable device 310. Furthermore, the adhesive portion 322 may include woven fabric, plastic or latex. The adhesive portion 322 may include at least one surface coated with an adhesive material. The adhesive portion 322 may be operable to keep the skin area under the main body portion 324 free from dirt, dust, abrasion, etc. Before mounting to the body, the adhesive portion 322 may be coupled to a disposable backing material, similar to an adhesive bandage, e.g. BAND-AID. Additionally or alternatively, the body-mountable device 310 may be adhered to the skin surface of the living body via a skin bonding material such a cyanoacrylate or a liquid bandage.

FIG. 3B illustrates a scenario 340 including body-mountable device 310 according to an example embodiment. In the illustrated scenario 340, the body-mountable device 310 may be adhered or otherwise mounted to a skin surface of a living body 342, e.g. a forearm. The living body 342 includes one or more lumens 346, which may be part of a circulatory system. In an example embodiment, lumen 346 may include an artery, a vein, or another type of blood vessel. The circulatory system could be part of a cardiovascular system of a human. However, other types of circulatory systems are contemplated. For example, the body-mountable device 310 may interact with other systems involving fluid flow.

As described in reference to FIG. 3A, the body-mountable device 310 may be configured to obtain optical information in the form of a photoplethysmogram (PPG). For example, the optical information may relate to a volume of the one or more lumens 346 and/or the blood contained therein. Namely, a PPG may measure and/or monitor volumetric changes in arteries or arterioles from blood perfusion near the surface of the skin.

In another example embodiment, the body-mountable device 310 may be configured to sense information relating to an analyte 344 within the lumen 346. Optionally, the living body 342 may include functionalized particles, which may be introduced into the circulatory system. In an example embodiment, the functionalized particles may be added to the bloodstream via injection, inhalation, and/or ingestion. In some embodiments, the body-mountable device 310 may be configured to detect a flow rate, concentration, or other information relating to the functionalized particles.

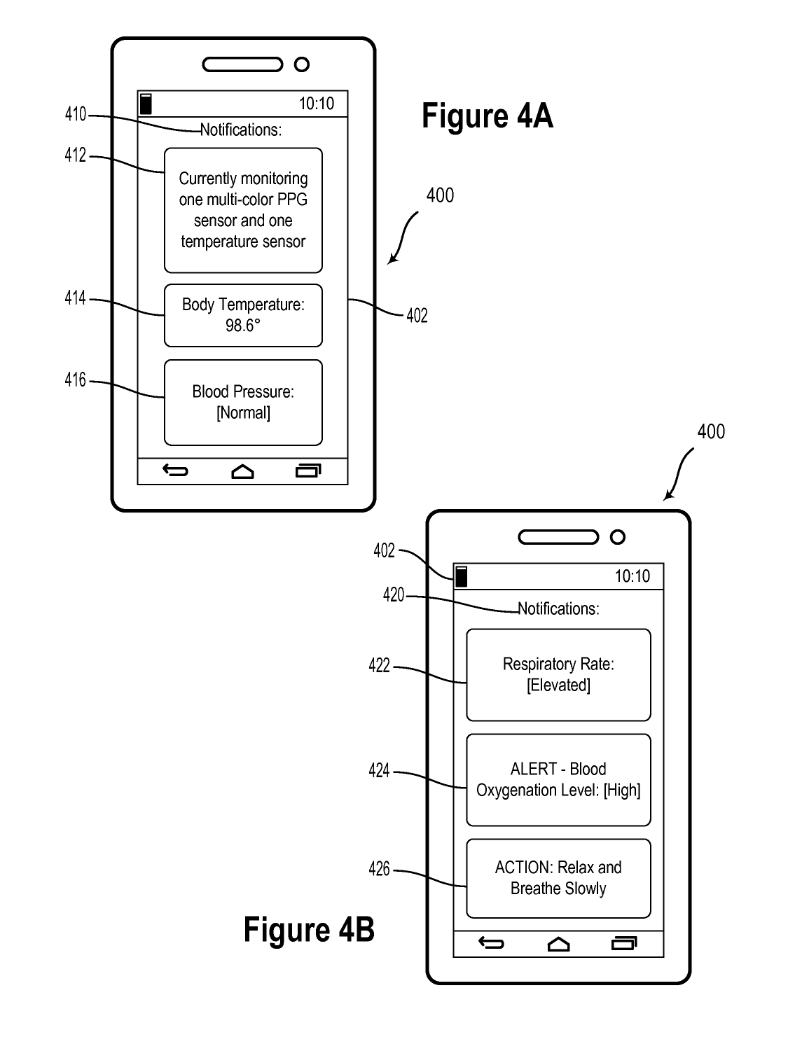

FIG. 4A illustrates a mobile device 400 according to an example embodiment. The mobile device 400 may be similar or identical to the mobile computing device 130 illustrated and described in reference to FIG. 1. Namely, the mobile device 400 may be a smartphone, laptop, tablet, e-reader, or another type of handheld computing device. The mobile device 400 includes a display 402. The mobile device 400 may include one or more buttons (not illustrated), icons, and/or touch-sensitive surfaces.

The mobile device 400 may be communicatively coupled to any of the body-mountable devices described herein. For example, the mobile device 400 may be coupled via a BLUETOOTH communication link with body-mountable devices 110, 200, or 310 as illustrated and described in reference to FIGS. 1, 2, 3A, and 3B.

The mobile device 400 may provide various notifications 410 via the display 402 that may relate to information from the communicatively-coupled body-mountable device. For example, the mobile device 400 may be operable to provide a monitoring notification 412 that may relate to the number and types of sensors being monitored. Specifically, the monitoring notification 412 may state “Currently monitoring one multi-color PPG sensor and one temperature sensor”.

Furthermore, the mobile device 400 may be configured to provide a temperature notification 414. The temperature notification 414 may provide information about an individual's body temperature. Additionally, the mobile device 400 may provide a blood pressure notification 416. The blood pressure notification 416 may provide a result from a comparison between an inferred blood pressure from the multi-color PPG sensor and a look-up table that includes blood pressure information based, for example, on height, weight, age, and/or sex.

FIG. 4B illustrates a mobile device 400 according to an example embodiment. FIG. 4B may illustrate further notifications 420 that may be provided via the display 402 of the mobile device 400. For example, a respiratory rate notification 422 may include a comparison between the inferred, measured, and/or calculated respiratory rate with a look-up table that includes respiratory rate information based on, for example, weight, age, and/or sex.

The notifications 420 may include alerts and/or warnings relating to physiological parameters. For example, the mobile device 400 may be configured to provide an alert 424 related to an elevated blood oxygenation level. Such an alert 424 may be provided to a user of the mobile device 400 and/or the wearer of the body-mountable device so as to avoid a hyperventilation state.

The notifications 420 may include instructions or advice relating to physiological parameters. For instance, the mobile device 400 may be configured to provide an action notification 426. The action notification 426 may relate to alert 424, however other types of physiological parameters, physiological conditions, and/or measurements may trigger action notifications. In an example embodiment, the action notification 426 may provide a prompt or suggestion for a user of the mobile device 400 to perform an action. In the above scenario, the action notification 426 may state “Relax and Breathe Slowly”. It is understood that many other types of general, action, and alert notifications are possible within the context of this disclosure. All such notifications are contemplated herein.

### Method Examples

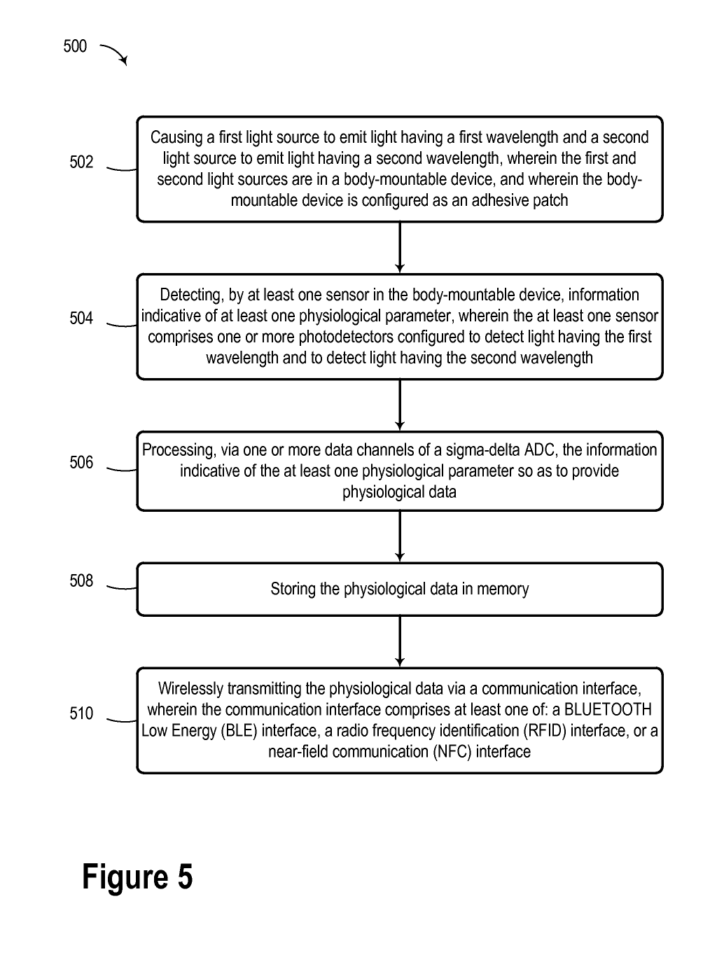

FIG. 5 illustrates a method 500, according to an embodiment. The method 500 includes blocks that may be carried out in any order. Furthermore, various blocks may be added to or subtracted from method 500 within the intended scope of this disclosure. The method 500 may correspond to steps that may be carried out using any or all of the devices and/or systems illustrated and described in reference to FIG. 1, 2A, 3A, 3B, 4A, or 4B.

Block 502 includes causing a first light source to emit light having a first wavelength and a second light source to emit light having a second wavelength. The first and second light sources are in a body-mountable device. The body-mountable device is configured as an adhesive patch. The body-mountable device described in the method could be body-mountable device 110, 200, or 310 as illustrated and described in reference to FIGS. 1, 2, and 3A, respectively. As described elsewhere herein, the first light source and the second light source may include light-emitting diodes (LEDs) or other light-emitting devices, such as one or more laser diodes.

Block 504 includes detecting, by at least one sensor in the body-mountable device, information indicative of at least one physiological parameter. The at least one sensor includes one or more photodetectors configured to detect light having the first wavelength and to detect light having the second wavelength. A combination of the multiple light sources and the one or more photodetectors may be operable to provide photoplethysmogram data relating to volumetric measurements of a lumen of subsurface vasculature.

Block 506 includes processing, via one or more data channels of a sigma-delta analog to digital converter (ADC), the information indicative of the at least one physiological parameter so as to provide physiological data. As described elsewhere herein, the sigma-delta ADC may convert or encode an analog amplitude of a photodetector signal into a pulse code modulated signal with the pulse frequency being related to the amplitude of the photodetector signal.

Block 508 includes storing the physiological data in memory. The memory may be a static random access memory (SRAM) chip, however many other types of memory devices are contemplated herein. In some embodiments, a time period of physiological data may be limited based on sensor sample rate, the number of sensors sampled, and/or memory size. In such a scenario, the most recent data collected may overwrite the oldest data.

Block 510 includes wirelessly transmitting the physiological data via a communication interface. The physiological data may be transmitted to a mobile computing device, such as mobile computing device 130 as illustrated and described in reference to FIG. 1. The communication interface may include at least one of: a BLUETOOTH Low Energy (BLE) interface, a radio frequency identification (RFID) interface, or a near-field communication (NFC) interface. In some examples, the physiological data may include raw data from the various sensors of the body-mountable device. In other example embodiments, the body-mountable device may at least partially process the raw sensor data before wireless transmission.

The wireless transmission of physiological data may be provided substantially continuously or may occur in response to a trigger. For example, in the case of an RFID communication interface, the wireless transmission of data may be triggered when a handheld reader, such as mobile communication device 130, comes into close proximity to the body-mountable device, e.g. within 2 meters. In other embodiments, the wireless transmission of physiological data may be triggered in response to a message from the mobile computing device 130. In yet further embodiments, the body-mountable device may wirelessly broadcast the physiological data.

The particular arrangements shown in the Figures should not be viewed as limiting. It should be understood that other embodiments may include more or less of each element shown in a given Figure. Further, some of the illustrated elements may be combined or omitted. Yet further, an illustrative embodiment may include elements that are not illustrated in the Figures.

While various examples and embodiments have been disclosed, other examples and embodiments will be apparent to those skilled in the art. The various disclosed examples and embodiments are for purposes of illustration and are not intended to be limiting, with the true scope being indicated by the following claims.

What is claimed is:

1. A system comprising:
a body-mountable device configured as an adhesive patch, the body-mountable device comprising:
a first light source and a second light source, wherein the first light source is configured to emit light having at least a first wavelength and the second light source is configured to emit light having at least a second wavelength;
at least one sensor, wherein the at least one sensor comprises one or more photodetectors configured to detect light having the first wavelength and to detect light having the second wavelength;
a battery;
a communication interface; and
a controller comprising a memory and a processor, wherein the memory stores instructions that are executable by the processor to cause the controller to perform operations comprising:
causing the first light source and the second light source to emit light;
receiving information from the at least one sensor, wherein the information is indicative of at least one physiological parameter; and
transmitting, via the communication interface, the information indicative of at least one physiological parameter; and
a mobile computing device configured to:
receive the transmitted information indicative of at least one physiological parameter.

2. The system of claim 1, wherein the body-mountable device is configured to adhere to a skin layer of a living body.

3. The system of claim 1, wherein the at least one sensor further comprises an accelerometer, wherein receiving information from the at least one sensor comprises receiving at least a portion of the information from the accelerometer.

4. The system of claim 1, wherein the at least one sensor further comprises a temperature sensor, wherein receiving information from the at least one sensor comprises receiving at least a portion of the information from the temperature sensor.

5. The system of claim 1, wherein the at least one sensor further comprises a piezoresistor, wherein receiving information from the at least one sensor comprises receiving at least a portion of the information from the piezoresistor.

6. The system of claim 1, wherein the at least one physiological parameter comprises at least one of: a heart rate, a blood pressure, or a blood oxygenation level.

7. The system of claim 1, wherein the first wavelength is within a range between 750 nanometers and 1 millimeter and the second wavelength is within a range between 620 nanometers and 750 nanometers.

8. The system of claim 1, wherein receiving information from the at least one sensor comprises receiving at least 120 samples per second from the at least one sensor.

9. The system of claim 1, wherein the communication interface comprises at least one of: a BLUETOOTH Low Energy (BLE) interface, a radio frequency identification (RFID) interface, or a near-field communication (NFC) interface.

10. The system of claim 1, wherein the controller further comprises a sigma-delta analog-to-digital converter (ADC) having one or more data channels.

11. A body-mountable device configured as an adhesive patch, the body-mountable device comprising:
a first light source and a second light source, wherein the first light source is configured to emit light having at least a first wavelength and the second light source is configured to emit light having at least a second wavelength;
at least one sensor, wherein the at least one sensor comprises one or more photodetectors configured to detect light having the first wavelength and to detect light having the second wavelength;
a battery;
a communication interface, wherein the communication interface comprises at least one of: a BLUETOOTH Low Energy (BLE) interface, a radio frequency identification (RFID) interface, or a near-field communication (NFC) interface; and
a controller comprising a memory, a processor, and a sigma-delta analog-to-digital converter (ADC) having one or more data channels, wherein the memory stores instructions that are executable by the processor to cause the controller to perform operations comprising:
causing the first light source and the second light source to emit light;
receiving information from the at least one sensor, wherein the information is indicative of at least one physiological parameter;
processing, via the one or more data channels of the sigma-delta ADC, the information indicative of the at least one physiological parameter so as to provide physiological data;
storing the physiological data in the memory; and
wirelessly transmitting the physiological data via the communication interface.

12. The body-mountable device of claim 11, wherein the body-mountable device is configured to adhere to a skin layer of a living body.

13. The body-mountable device of claim 11, wherein the at least one sensor further comprises an accelerometer, wherein receiving information from the at least one sensor comprises receiving at least a portion of the information from the accelerometer.

14. The body-mountable device of claim 11, wherein the at least one sensor further comprises a temperature sensor, wherein receiving information from the at least one sensor comprises receiving at least a portion of the information from the temperature sensor.

15. The body-mountable device of claim 11, wherein the at least one sensor further comprises a piezoresistor, wherein receiving information from the at least one sensor comprises receiving at least a portion of the information from the piezoresistor.

16. The body-mountable device of claim 11, wherein the at least one physiological parameter comprises at least one of: a heart rate, a blood pressure, or a blood oxygenation level.

17. A method comprising:
causing a first light source to emit light having a first wavelength and a second light source to emit light having a second wavelength, wherein the first and second light sources are in a body-mountable device, and wherein the body-mountable device is configured as an adhesive patch;
detecting, by at least one sensor in the body-mountable device, information indicative of at least one physiological parameter, wherein the at least one sensor comprises one or more photodetectors configured to detect light having the first wavelength and to detect light having the second wavelength;
processing, via one or more data channels of a sigma-delta analog-to-digital converter (ADC), the information indicative of the at least one physiological parameter so as to provide physiological data;
storing the physiological data in memory; and
wirelessly transmitting the physiological data via a communication interface, wherein the communication interface comprises at least one of: a BLUETOOTH Low Energy (BLE) interface, a radio frequency identification (RFID) interface, or a near-field communication (NFC) interface.

18. The method of claim 17, wherein the at least one physiological parameter comprises at least one of: a heart rate, a blood pressure, or a blood oxygenation level.

19. The method of claim 17, wherein the at least one sensor further comprises an accelerometer, wherein at least a portion of the information indicative of at least one physiological parameter is detected by the accelerometer.

20. The method of claim 17, wherein the at least one sensor further comprises a temperature sensor, wherein at least a portion of the information indicative of at least one physiological parameter is detected by the temperature sensor.
